(19) United States
(12) Patent Application Publication
(10) Pub. No.: US 2017/0128099 A1
(43) Pub. Date: May 11, 2017

(54) APPARATUS FOR PROVIDING AND MAINTAINING ACCESS TO A SURGICAL SITE

(71) Applicant: OXFORD UNIVERSITY INNOVATION LIMITED, Oxford (GB)

(72) Inventors: Zhong You, Oxford, Oxfordshire (GB); Puneet Plaha, Oxford, Oxfordshire (GB); Jiayao Ma, Oxford, Oxfordshire (GB); Simon Cudlip, Oxford, Oxfordshire (GB)

(73) Assignee: OXFORD UNIVERSITY INNOVATION LIMITED, Botley (GB)

(21) Appl. No.: 15/322,443

(22) PCT Filed: Jun. 23, 2015
(86) PCT No.: PCT/GB2015/051822

§ 371 Date: Dec. 27, 2016

(30) Foreign Application Priority Data
Jun. 27, 2014 (GB) 1411486.2

(51) Int. Cl. A61B 17/34; A61B 17/02

(52) U.S. Cl. CPC A61B 17/3439; A61B 17/0218; A61B 2017/00867

(57) ABSTRACT

The invention relates to an apparatus for providing and maintaining access to a surgical site, particularly a surgical site that needs to be accessed through delicate tissue such as brain tissue. The invention relates particularly to an apparatus that uses an expandable member or trochar. In one arrangement the expandable member is positioned proximally relative to the distal end of an obturator when the apparatus is in an insertion configuration for insertion to the surgical site. The expandable member comprises a frame and a membrane attached to the frame. The frame and membrane are configured to allow bending of at least a portion of the surface of the expandable member about an axis perpendicular to the longitudinal axis without breakage of the expandable member.

### FIELD

The invention relates to an apparatus for providing and maintaining access to a surgical site, particularly a surgical site that needs to be accessed through delicate tissue such as brain or spinal tissue. The invention relates particularly to an apparatus that uses an expandable member or trochar.

### BACKGROUND

Access to surgical sites in the brain is problematic because of the need to minimise damage to the surrounding tissue. Devices are known which enable a cylindrical tube to be inserted to the surgical site. Once installed a surgeon can then access the surgical site through the tube with less risk of surgical instruments causing damage to tissue that is outside of the tube. The tube is rigid and provides protection to the surrounding tissue.

The tube can be inserted in a number of different ways. Generally, a tapered member is provided at a leading end of the device as the device is inserted into the brain tissue. The tapered member helps to push tissue aside rather than cutting through the tissue and thereby reduces damage caused by the insertion process. The tube follows the tapered member to the surgical site. The tapered member can be withdrawn through the tube, when the tube is in position, to allow access to the surgical site through the tube.

The circumference of the tube needs to be large enough to allow the required surgical operations to be carried out effectively. However, larger tubes are more difficult to insert into the brain without causing too much damage. Furthermore, the rigid nature of the tubes makes it difficult to access regions which are slightly outside of the diameter of the tube, which tends to increase the minimum circumference of the tube that is required for a given operation.

An object of the present invention is to provide an improved way of providing and maintaining access to a surgical site in a delicate region of the body which at least partially addresses one or more of the problems with the prior art discussed above.

### SUMMARY

According to an aspect of the invention, there is provided an apparatus for providing and maintaining access to a surgical site, comprising: an obturator having a distal end that is tapered in a direction parallel to a longitudinal axis of the apparatus; and an expandable member positioned proximally relative to the distal end of the obturator when the apparatus is in an insertion configuration for insertion to the surgical site, wherein: the expandable member comprises a frame and a membrane attached to the frame, the frame and membrane being configured to allow the expandable member to be expandable from a radially-contracted state to a radially-expanded state, the radial direction being perpendicular to the longitudinal axis; the frame is configured to resist radial compression when in the radially-expanded state; and the frame and membrane are configured to allow bending in response to an externally applied force, about an axis perpendicular to the longitudinal axis, of at least a portion of the surface of the expandable member when the expandable member is in the radially-expanded state, without breakage of the expandable member.

The apparatus allows a passage to the surgical site to be formed by an expandable member, which allows the cross-section of the apparatus during insertion to be made smaller, thus reducing the risk of tissue damage during insertion. Combining this property with the capacity for portions of the expandable member's surface to bend about an axis perpendicular to the longitudinal axis increases the range of angles at which surgical devices can be inserted through the expandable member, thus increasing the range of positions at the surgical site that are accessible for a given average cross-sectional area of the expandable member. The average cross-sectional area of the expandable member can thus be made smaller relative to devices allowing the same degree of access to the surgical site, further reducing the risk of damage to tissue caused by the presence of the expandable member.

In an embodiment the expandable member comprises a frame attached to a membrane. The membrane seals the inside of the expandable member from the tissue outside of the expandable member and protects the tissue. When the membrane is positioned radially inwardly of the frame, the membrane may reduce the risk of interference between surgical devices inserted into the expandable member and the frame (e.g. snagging or catching of the surgical devices against the frame). When the membrane is positioned radially outwardly of the frame, the frame may improve the smoothness of the structure that presses outwards against the delicate tissue through which the apparatus has been inserted, thereby reducing the risk of damage to that tissue. Preferably a membrane is provided on both the radially inner and radially outer sides of the frame to achieve all of the advantages mentioned above. The frame may also be partially or completely encapsulated by the membrane.

In an embodiment, either or both of the radially-expanded state and the radially-contracted state has/have non-circular cross-sections, preferably substantially oval or elliptical. Thus, a smooth shape that is relative elongate in one direction is provided. Such a shape may be inserted through natural folds in the brain tissue more efficiently than shapes which are less elongate. If the direction of elongation is aligned along the direction of natural folds in the brain tissue the risk of damage to the tissue caused by the expansion process and/or prolonged maintenance of the expanded member 8 in the radially-expanded state may be reduced.

### BRIEF DESCRIPTION OF THE DRAWINGS

Embodiments of the invention will now be described, by way of example only, with reference to the accompanying drawings in which corresponding reference symbols indicate corresponding parts, and in which:

FIG. 1A is a schematic side sectional view of an apparatus for providing and maintaining access to a surgical site according to an embodiment;

FIG. 1B is a schematic end sectional view of the apparatus of FIG. 1A;

FIG. 2 shows expansion of an expandable member in the apparatus of FIGS. 1A and 1B;

FIG. 3 shows withdrawal of an obturator after expansion of the expandable member;

FIG. 4 is a schematic perspective view of an example expandable member in a radially-contracted state;

FIG. 5 is a depicts the expandable member of FIG. 4 in a radially-expanded state, caused in this example by axial compression;

FIG. 6 is a schematic side sectional view of an apparatus for providing and maintaining access to a surgical site according to an alternative embodiment, with the expandable member in a radially-contracted state;

FIG. 7 shows the apparatus of FIG. 6 with the expandable member in a radially-expanded state, driven in this embodiment by expansion of a balloon;

FIG. 8 shows the apparatus of FIGS. 6 and 7 illustrating withdrawal of the obturator through the expandable member in the radially-expanded state;

FIG. 9 is a schematic view of an unwrapped expandable member showing an example frame and membrane;

FIG. 10 is a schematic end sectional view of an expandable member comprising a frame sandwiched between two membranes;

FIG. 11 illustrates an example bending mode of an expandable member;

FIG. 12 illustrates a further example bending mode of the same or a different expandable member;

FIG. 13 illustrates a further example bending mode of the same or a different expandable member;

FIG. 14 depicts an expandable member having an oval cross-sectional shape in either or both of the radially-expanded and radially-contracted states;

FIG. 15 depicts an alternative expandable member having an elongate cross-sectional shape in either or both of the radially-expanded and radially-contracted states;

FIG. 16 is a schematic view of an unwrapped expandable member showing a further example frame and membrane; and

FIG. 17 is a schematic view of an unwrapped expandable member showing a further example frame and membrane.

### DETAILED DESCRIPTION

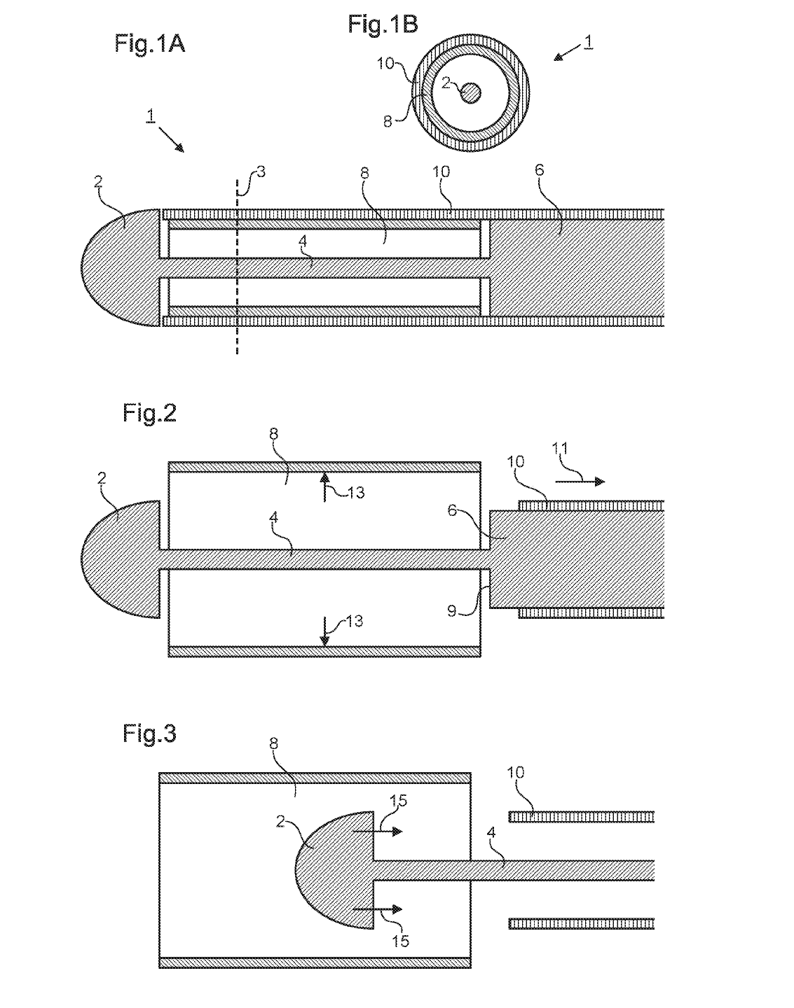

In an embodiment there is provided an apparatus for providing and maintaining access to a surgical site. The surgical site may be within the brain or spinal cord for example The apparatus is particularly suitable for providing access to surgical sites with a minimum of damage to tissue in between a point of access to the body (e.g. a hole in the skull) and the surgical site (e.g. a tumour within the brain). An example of such an apparatus is depicted in FIGS. 1-3.

FIG. 1A is a schematic side sectional view of the apparatus 1. FIG. 1B is a schematic end sectional view of the apparatus of FIG. 1A along broken line 3. The apparatus 1 comprises an obturator having a distal end 2 that is tapered in a direction parallel to a longitudinal axis of the apparatus 1. In the embodiment shown the tapered distal end 2 is connected to an inner shaft 6, positioned proximally relative to the distal end 2, via connection element 4. The apparatus 1 comprises an expandable member 8 positioned proximally relative to the distal end 4 of the obturator when the apparatus is in an insertion configuration for insertion to the surgical site. The expandable member 8 comprises a frame and a membrane attached to the frame. In the particular embodiment shown the expandable membrane 8 takes the form of a hollow cylinder, but this is not essential. The frame and membrane are expandable from a radially-contracted state to a radially-expanded state, the radial direction being perpendicular to the longitudinal axis.

FIGS. 1A and 1B show the apparatus 1 in the insertion configuration. A characteristic of the insertion configuration is that it is radially compact and therefore suitable for insertion to a surgical site. In this configuration, the expandable member 8 is in the radially-contracted state. In the embodiment of FIGS. 1-3 the expandable member 8 is self-expanding. This may be achieved for example by arranging for the frame to be self-expanding and the membrane to be stretchable. The expandable member 8 is held in the radially-contracted state in the insertion configuration by an outer sleeve 10 positioned radially outside of the expandable member 8 when the apparatus 1 is in the insertion configuration. The outer sleeve 10 thus prevents premature expansion, which might otherwise occur for heat induced shape memory devices when they reach body temperatures or for mechanically restrained or stress induced shape memory devices. The outer sleeve 10 may be configured to be slidable in a longitudinal direction in order to allow the expandable member 8 to be released when the distal end 2 has reached the surgical site. This process is illustrated in FIGS. 2 and 3.

FIG. 2 shows how longitudinal withdrawal (arrow 11) of the outer sleeve 10 leads to removal of the radial constraint on the self-expanding expandable member 8, which then duly expands radially (arrows 13) to the radially-expanded state. The inner shaft 6 comprises a distally facing shoulder portion 9 that prevents proximal movement of the expandable member 8 while the outer sleeve 10 is being withdrawn. The inner shaft 6 may be slidably engaged inside the outer sleeve 10 to allow the relative longitudinal movement between the inner shaft 6 and the outer sleeve 10.

FIG. 3 shows how the obturator can be pulled (arrows 15) through the expandable member 8 in a proximal direction and thereby removed from the apparatus 1 when the expandable member 8 is in the radially-expanded state after expansion of the expandable member 8.

The self-expanding property of the expandable member 8 of the embodiment of FIGS. 1-3 may be achieved in a variety of manners. For example, the frame of the expandable membrane 8 may be formed from a shape memory material (e.g. shape memory alloy).

In other embodiments the expandable member 8 requires application of an external force to at least partially drive the transition from the radially-contracted state to the radially-expanded state. In embodiments of this type an expander may be provided for driving the transition from the radially-contracted state to the radially-expanded state.

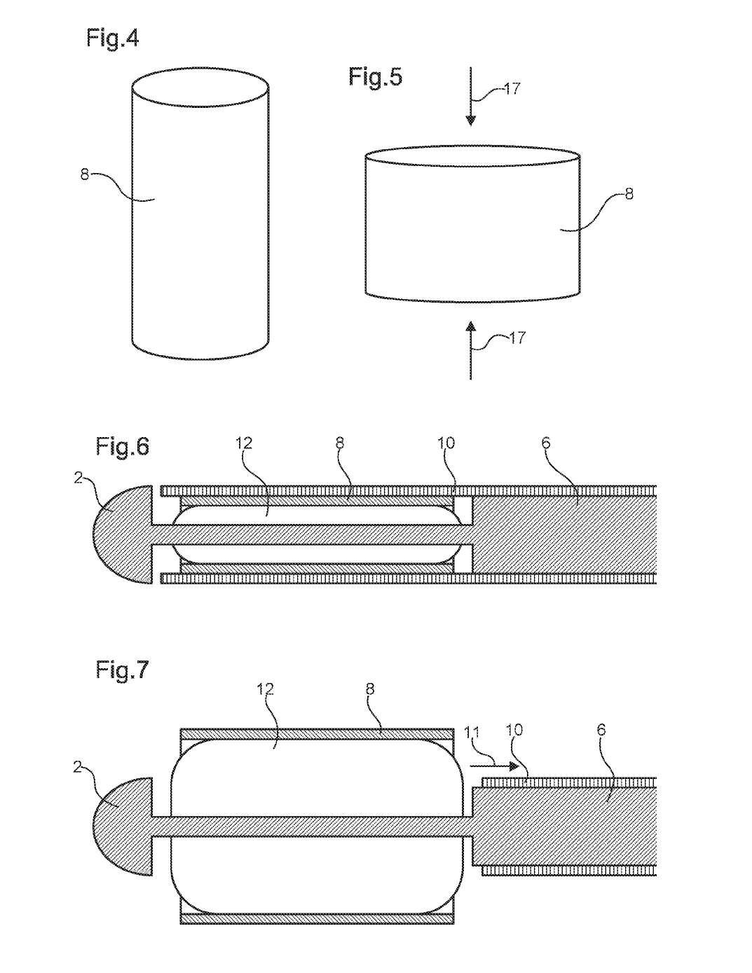

FIGS. 4 and 5 depict an expandable member 8 according to such an embodiment in which transition between the radially-contracted and radially-expanded states is achieved by controlling the axial length of the expandable member 8. In an example of this type of embodiment, the frame is configured to expand radially when compressed longitudinally and to be compressed radially when extended longitudinally. An expander (not shown) may be provided to compresses the frame longitudinally (and thereby indirectly cause radial expansion) or to push the frame out radially directly (e.g. using a balloon). FIG. 4 shows the expandable member 8 in the radially-contracted state. FIG. 5 shows the expandable member 8 in the radially-expanded state (after longitudinal compression in the direction of arrows 17). An expandable member of this type may be formed using an interwoven frame for example, which may be self expanding if formed from a shape memory material (in which case the expander may not be required).

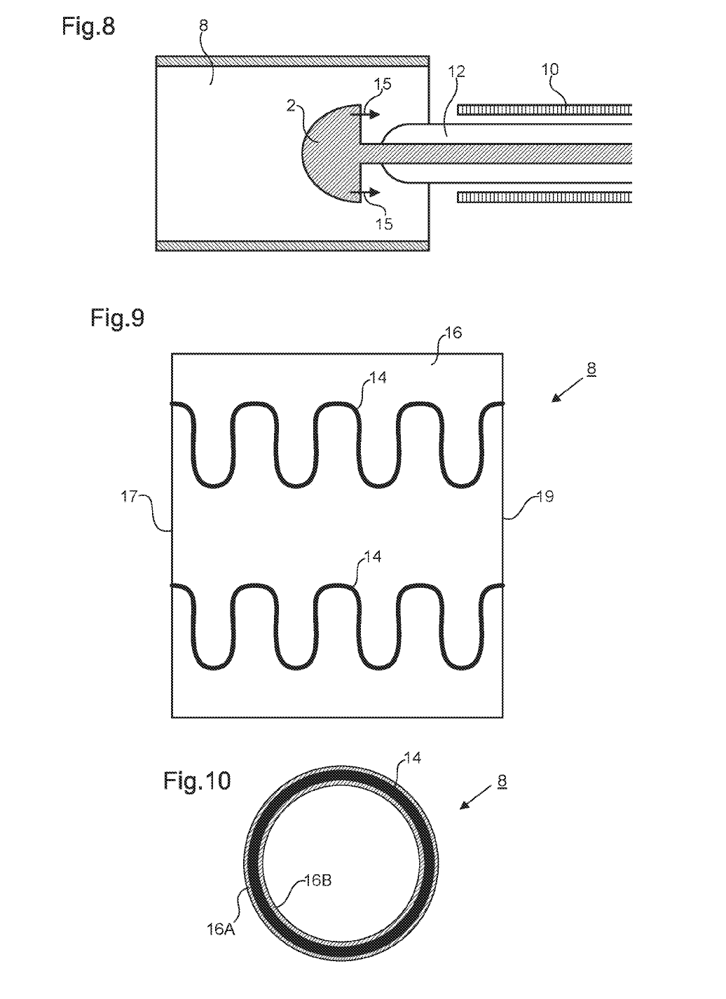

FIGS. 6-8 illustrate an embodiment in which the expander comprises a balloon 12. FIG. 6 is a schematic side sectional view of the apparatus 1 with the expandable member 8 in a radially-contracted state. As in the arrangement of FIG. 1A, the expandable member 8 may be protected radially by an outer sleeve 10 that may be slidably engaged with the apparatus 1 on a radially outer surface thereof. FIG. 7 is a schematic side sectional view of the apparatus with the expandable member in a radially-expanded state, driven by expansion of the balloon 12 (after proximal withdrawal of the outer sleeve 10; arrow 11). FIG. 8 is a schematic side sectional view of the apparatus 1 showing withdrawal of the obturator through the expandable member 8 (arrows 15) after deflation of the balloon 12. In this embodiment the deformation of the expandable member 8 during expansion is at least partially plastic so that the expandable member 8 does not radially contract when the balloon 12 is deflated.

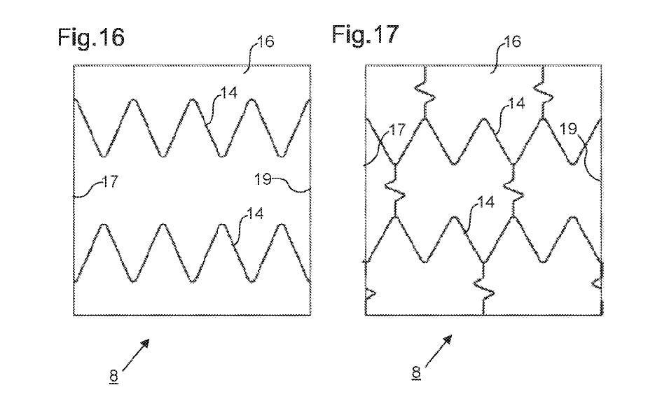

FIG. 9 depicts how an example expandable member 8 may look if unwrapped from a “wrapped” (e.g. cylindrical) form to a planar form. Thus, in the wrapped state the leftmost edge 17 would be in contact (or integral with) the rightmost edge 19 of the expandable member 8. Alternative example configurations are shown in FIGS. 16 and 17. In embodiments of this type the frame 14 may comprise a plurality of rings (two are provided in the particular example shown). Each ring forms a closed loop around the longitudinal axis (in the wrapped state). The rings have an undulating form in the circumferential direction. In other embodiments the rings may have other forms. The rings may be annular or may zigzag along the circumferential direction. The zigzags in direction may or may not be aligned with each other in the longitudinal direction. In other embodiments the frame may comprise a single ring or more than two rings. In the embodiment shown the rings are all disconnected from each other, but this is not essential. In other embodiments two or more of the rings may be connected to each other. The rings may be formed from a shape memory alloy such as Nitinol or a material having plastic properties such as stainless steel.

In an embodiment, the membrane 16 comprises a fabric that is connected to the frame 14 using stitching. Alternatively, the membrane 16 may comprise a polymer such as PTFE that is coated onto a skeleton formed by the frame 14. The membrane 16 may be stretchable to allow the transition from the radially-contracted state to the radially-expanded state. Alternatively or additionally, the membrane 16 may be folded in the radially-contracted state and unfold to form to the radially-expanded state.

In an embodiment, the membrane 16 is positioned radially inside of the frame 14. This would be achieved in an embodiment of the type shown in FIG. 9 by wrapping the membrane in a direction going into the page. In an alternative embodiment, the membrane 16 is positioned radially outside of the frame 14. This would be achieved in an embodiment of the type shown in FIG. 9 by wrapping the membrane in a direction going out of the page.

Alternatively, a first membrane and a second membrane may be provided with the frame 14 positioned in between. An example of such an arrangement is shown in the schematic end sectional view of FIG. 10. Here, a first membrane 16A is positioned radially outside of the frame 14 and a second membrane 16B is positioned radially inside of the frame 14.

Positioning a membrane 16A radially outside of the frame 14 helps to prevent damage to tissue outside of the expandable member 8 during expansion of the expandable member 8 and/or while the expandable member 8 is maintained in the expanded state during a surgical procedure. The membrane helps provide a smoother external surface than would be provided if the frame were exposed directly to the tissue.

Positioning a membrane 16B radially inside of the frame 14 helps to prevent interference with the insertion or manipulation of surgical device within the expandable member 8 during a surgical procedure. The membrane helps to provide smoother internal surface than would be provided if the frame were exposed. The risk of damage to surgical devices and/or damage to tissue caused by incorrect insertion or manipulation of surgical devices is thereby reduced.

In an embodiment, the frame 14 may be at least partially (i.e. partially or fully) encapsulated within the membrane 16. For example at least portions of the frame 14 may be encapsulated in such a way as to be covered by material of the membrane 16 on radially inner and radially outer surfaces of the frame 14. Encapsulating the frame 14 tends to increase the smoothness of interior and/or exterior surfaces of the expandable member 8, thereby reducing risk of damage to surgical devices and/or tissue during use, as discussed above.

In any of the embodiments disclosed the frame may be configured to resist radial compression when in the radially-expanded state. Thus, the frame is stable in the radially-expanded state and does not require any external support in order to stay in the radially-expanded state. The frame can resist typical inward pressures exerted by the tissue which has been pushed aside by insertion of the apparatus 1.

The use of shape memory materials has been discussed above. Usually such materials are used in an elastic regime (so that deformations are reversible). The expandable member 8 (e.g. the frame thereof) may also comprise elastic materials that are not shape memory materials and which are still operated in an elastic regime. However, operation in an elastic regime is not essential. In other embodiments, the frame 14 may be configured to deform plastically during the expansion from the radially-contracting state to the radially-expanded state. The plastic deformation may assist with providing the resistance to radial compression required in the radially-expanded state.

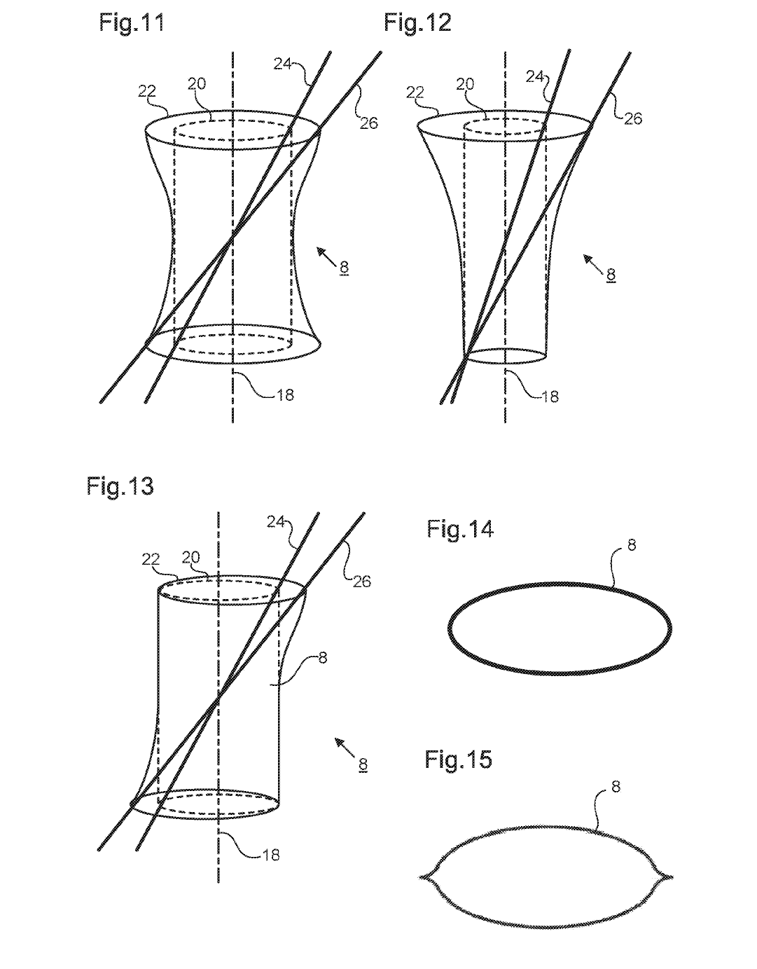

In any of the embodiments disclosed one or more portions of the frame and membrane may be configured to allow bending, preferably but not exclusively reversible (e.g. elastic) bending, about an axis perpendicular to the longitudinal axis. FIGS. 11-13 illustrate example bending modes of the expandable member 8 in the radially-expanded state. In each figure, broken lines 20 illustrate a form taken by the expandable member 8 before bending of any portion of the expandable member and solid lines 22 illustrate a form taken by the expandable member 8 after bending of one or more portions of the expandable member. In this context, bending is understood to mean a change in the curvature of at least a portion of the surface of the expandable member 8 and is not limited for example to uniform bending of the whole expandable member 8. The bending may arise due to external forces applied to the expandable member 8 rather than any internal forces due to elastic properties of the frame or membrane. The external forces may be applied by a surgeon manipulating or inserting a surgical device through the expandable member 8 for example. As mentioned above, the frame and membrane are configured to allow bending of at least a portion of the surface of the expandable member 8 along at least a portion of the length of the expandable member 8 about an axis perpendicular to the longitudinal axis 18. In the examples shown it can be seen that portions of the extreme left and right edges of the expandable member 8 after bending are curved in the plane of the page due to the bending. The curvature may be described locally by reference to a radius of curvature about an axis that is perpendicular to the longitudinal axis 18 (and perpendicular to the plane of the page in the case of the curvature at the extreme left and right edges of the expandable member 8).

The bending of at least a portion of the surface of the expandable member comprises a decrease in the radius of curvature to 1 m or less without breakage of the expandable member and/or elastically.

Configuring the expandable member 8 to allow such bending of the surface increases the range of angles available for insertion and manipulation of surgical devices through the expandable element 8 during use without requiring tilting of the whole expandable member 8. This is illustrated by the thick solid lines 24 and 26. Line 24 represents a maximum angle (“access angle”) of a notional linear device without tilting or deformation of the expandable member 8. Line 26 represents a maximum angle (“access angle”) of the same device allowed by bending according to an embodiment (but no tilting). In prior art arrangements in which a rigid tube is used, the angle of line 26 could only be achieved by tilting the whole tube. Such a tilting of the tube would impose significantly higher stresses on tissue outside of the tube than would the localised deformation of the expandable member 8 associated with the bending that achieves the same access angle. Increasing the access angle makes it possible for surgical devices to reach areas which are outside of a radial width of the expandable member and/or make it easier to access areas which are near the radial limit of the expandable member 8. The average width of the expandable member 8 can therefore be made smaller for the same degree of access, which will tend to reduce the risk of damage to brain tissue during insertion (because the overall device can be made narrower) and during operation after the expandable member has been expanded to the radially-expanded state.

FIG. 11 shows an example where the surfaces at the proximal (top) and distal (bottom) ends of the expandable member 8 are bent outwards to provide large cross-sectional areas in these regions. FIG. 12 shows an example where this is done at a proximal (top) end of the expanded member 8 but not at the distal end (bottom). Distortion of the expandable member at a proximal end may be preferable to distortion at a distal end because it may provide a useful increase in accessibility to the surgical site for the surgeon with a minimum of tissue damage risk.

In FIGS. 11 and 12 the bending is applied in a rotationally symmetric manner relative to the longitudinal axis, but this is not essential. FIG. 13 shows an embodiment in which bending is asymmetric. Here, the same angle of access is achieved as that of FIG. 11 with less overall distortion (and therefore less risk of tissue damage caused by the distortion). These modes of bending are only examples. Many other modes are possible, either by a single expandable member or by different expandable members. The frame and/or membrane can be adapted to allow particular types of bending according to the clinical needs of the particular type of surgical operation envisaged. The frame and/or membrane may be adapted so that a radial stiffness varies longitudinally and/or circumferentially over the surface of the expandable member 8 in order to support the bending deformations that are needed. For example, bending deformations of the type shown in FIG. 11 or 13 are required, it may be desirable to arrange for the radial stiffness to be lower at proximal and/or distal ends of the expandable member 8 in comparison with regions that are more longitudinally central. This allows the expandable element 8 to provide firm support to the surrounding tissue while at the same time allowing a wide range of access angles. Where bending deformations of the type shown in FIG. 12 are required, it may be desirable to arrange for the radial stiffness to be lower at the proximal end of the expandable member 8 relative to regions that are more distal.

In the embodiments discussed above it is envisaged that the cross-sectional area of the expandable member 8 in the radially-expanded but not yet bent or deformed state (e.g. prior to any external force being applied, such as a surgeon pressing the shaft of a surgical device against the inside of the expandable element 8) will be substantially uniform (as shown by the broken lines 20 in FIGS. 11-13). However, this is not essential. In other embodiments, the expandable member 8 may be configured to expand into a state having a non-uniform cross-section even before any external forces have been applied. For example, the expandable member 8 may be configured to expand into a state in which the cross-sectional area is larger at proximal and distal ends than in regions that are more central longitudinally, similar to the shape shown by solid lines 22 in FIG. 11. Alternatively, the expandable member 8 may be configured to expand into a state in which the cross-sectional area is larger at the proximal end than in regions that are distal from the proximal end, similar to the shape shown by the solid lines 22 in FIG. 12.

In the embodiments discussed above the expandable member 8 has been arranged to have a circular cross-section at substantially all longitudinal positions, both in the radially-expanded and radially-contracted states (in the absence of any bending of the surface caused by external forces). However, this is not essential. In other embodiments, either or both of the radially-expanded state and the radially-contracted state may have non-circular cross-sections along all or a portion of the longitudinal length of the expandable member 8. In examples of such an embodiment the non-circular cross-section is substantially oval (e.g. a flattened circle or at least a shape that has no sharp angles and which is longer in a first direction that in a second direction) or elliptical. Examples of such an expandable member 8 are shown schematically in FIGS. 14 and 15, which are views along the longitudinal direction of the expandable member 8 and may represent the expandable member 8 in either of the radially-expanded state and the radially-contracted state. Thus, a cross-section having a smooth shape that is relatively elongate in one direction may be used. Such a shape may be inserted through natural folds in the brain tissue more efficiently than shapes which are less elongate (e.g. circular) and with less damage to tissue. It may therefore be advantageous to arrange for the expandable member 8 to adopt such a shape at least in the radially-contracted state to facilitate insertion. In this case, the apparatus 1 may also comprise a tapered distal end 2, outer sleeve 10 and/or inner shaft 6 that have correspondingly shaped cross-sections. Additionally or alternatively the expandable member 8 may be configured to adopt the elongate (oval) cross-sectional shape when in the radially-expanded state. If the direction of elongation is aligned along the direction of natural folds in the brain tissue the risk of damage to the tissue caused by the expansion process and/or prolonged maintenance of the expandable member 8 in the radially-expanded state may be reduced.

1. An apparatus for providing and maintaining access to a surgical site, comprising:
an obturator having a distal end that is tapered in a direction parallel to a longitudinal axis of the apparatus; and
an expandable member positioned proximally relative to the distal end of the obturator when the apparatus is in an insertion configuration for insertion to the surgical site, wherein:
the expandable member comprises a frame and a membrane attached to the frame, the frame and membrane being configured to allow the expandable member to be expandable from a radially-contracted state to a radially-expanded state, the radial direction being perpendicular to the longitudinal axis;
the frame is configured to resist radial compression when in the radially-expanded state; and
the frame and membrane are configured to allow bending in response to an externally applied force, about an axis perpendicular to the longitudinal axis, of at least a portion of the surface of the expandable member when the expandable member is in the radially-expanded state, without breakage of the expandable member.

2. An apparatus according to claim 1, wherein the bending is reversible.

3. An apparatus according to claim 1, wherein the frame is a self-expanding frame.

4. An apparatus according to claim 1, further comprising an expander for driving the expandable member from the radially-contracted state to the radially-expanded state.

5. An apparatus according to claim 4, wherein the expander comprises a balloon.

6. An apparatus according to claim 4, wherein the frame is configured to expand radially when compressed longitudinally and the expander is configured to compress the frame longitudinally.

7. An apparatus according to claim 4, wherein the frame is configured to deform plastically during the expansion from the radially-contracted state to the radially-expanded state.

8. An apparatus according to claim 1, wherein the membrane is positioned radially inside of the frame.

9. An apparatus according to claim 1, wherein the membrane is positioned radially outside of the frame.

10. An apparatus according to claim 1, wherein the frame is at least partially encapsulated within the membrane.

11. An apparatus according to claim 1, wherein a first membrane and a second membrane are provided, the first membrane being positioned radially outside of the frame and the second membrane being positioned radially inside of the frame.

12. An apparatus according to claim 1, configured such that the obturator can be pulled through the expandable member in a proximal direction and thereby removed from the apparatus when the expandable member is in the radially-expanded state.

13. An apparatus according to claim 1, further comprising an outer sleeve positioned radially outside of the expandable member when the apparatus is in an insertion configuration for insertion to the surgical site.

14. An apparatus according to claim 13, further comprising an inner shaft slidably engaged inside the outer sleeve and configured to prevent longitudinal movement of the expandable member in the proximal direction during proximal withdrawal of the outer sleeve to deploy the expandable member.

15. An apparatus according to claim 1, wherein the frame comprises a plurality of rings, each ring forming a closed loop around the longitudinal axis.

16. An apparatus according to claim 15 wherein at least one of the rings is disconnected from all other rings.

17. An apparatus according to claim 1 wherein the frame comprises a shape memory material.

18. An apparatus according to claim 1, wherein either or both of the radially-expanded state and the radially-contracted state has/have non-circular cross-sections in the absence of bending caused by the externally applied force.

19. An apparatus according to claim 18, wherein the non-circular cross-section is substantially oval or elliptical.

20. An apparatus according to claim 1, wherein the bending of at least a portion of the surface of the expandable member comprises a decrease in the radius of curvature to 1 m or less.

21. (canceled)
